(19) United States
(12) Patent Application Publication
(10) Pub. No.: US 2026/0327176 A1
(43) Pub. Date: Oct. 22, 2026

(54) (METH)ACROLEIN AND/OR (METH)ACRYLIC ACID PRODUCTION WITH REDUCED AMOUNT OF HIGH BOILING POINT BYPRODUCT

(71) Applicant: Rohm and Haas Company, Collegeville, PA (US)

(72) Inventor: Jinsuo Xu, Berwyn, PA (US)

(21) Appl. No.: 19/113,134

(22) PCT Filed: Sep. 26, 2023
(86) PCT No.: PCT/US2023/033647

§ 371 Date: Mar. 19, 2025

Related U.S. Application Data
(60) Provisional application No. 63/410,644, filed on Sep. 28, 2022.

(51) Int. Cl. C07C 51/235; B01J 8/02; B01J 23/31; B01J 35/30; C07C 45/28

(52) U.S. Cl. CPC C07C 51/235; B01J 8/0278; B01J 23/31; B01J 35/394; C07C 45/28; B01J 2208/027

(57) ABSTRACT

A reactor for producing (meth)acrolein and/or (meth)acrylic acid from a reactant selected from propylene, isobutylene and tert-butanol. The reactor comprises an inlet for the reactant to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor has a molar ratio of bismuth to molybdenum that is lower than the catalyst closest to the outlet of the reactor.

### FIELD OF THE INVENTION

The present invention relates to a catalyst bed and process for the production of (meth)acrolein and/or (meth)acrylic acid.

### BACKGROUND OF THE INVENTION

(Meth)acrylic acid and its esters are important monomers for a variety of polymers including coatings, adhesives, and superabsorbent polymers. The majority of (meth)acrylic acid is produced via two-stage oxidation of isobutylene, tert-butanol, or propylene, in which the olefin or tert-butanol is first oxidized over a molybdenum (Mo) and bismuth (Bi) based mixed oxide catalyst to primarily form (meth)acrolein with (meth)acrylic acid being a minor product. The (meth)acrolein is then oxidized over a molybdenum and vanadium (V) and/or phosphorous (P) based mixed oxide catalyst to produce (meth)acrylic acid. Main byproducts of the acrylic acid production process from propylene include carbon monoxide, carbon dioxide, acetic acid, formaldehyde, acetaldehyde, propionic acid, propionaldehyde, acetone, allyl alcohol, maleic acid (or maleic anhydride), benzaldehyde, benzoic acid, furfural, and phthalic acids. In addition to these byproducts, methacrylic acid production processes may form other byproducts such as acrolein, acrylic acid, diacetyl, isobutyraldehyde, isobutyric acid, phthalic anhydride, and benzo tricarboxylic acids. These byproducts can be removed via distillation, extraction, melt crystallization, or combination of them. However, removal of these byproducts adds cost and complexity to the process.

U.S. Pat. No. 9,440,904 discloses a process for producing acrolein and/or acrylic acid, or methacrolein and/or methacrylic acid using two kinds of catalysts having different formulations that are stacked in an axial direction with higher productivity to (meth)acrolein. In the two kinds of catalysts, the component amount of bismuth relative to molybdenum decreases from the gas inlet side toward the gas outlet side of the reactor, and the component amount of iron relative to molybdenum increases from the gas inlet side toward the gas outlet side.

There is need for a catalyst bed and process that is capable of reducing the amounts of byproducts formed when (meth)acrolein and/or (meth)acrylic acid is produced from olefins or tert-butanol.

### SUMMARY OF THE INVENTION

The present invention is directed to a reactor and method for preparing (meth)acrolein and/or (meth)acrylic acid from an olefin or tert-butanol, and further, to produce (meth)acrylic acid from the (meth)acrolein.

According to one aspect of the present invention, a reactor for producing (meth)acrolein and/or (meth)acrylic acid comprises an inlet for an olefin or tert-butanol to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor has a molar ratio of bismuth to molybdenum that is lower than the catalyst closest to the outlet of the reactor.

Another aspect of the present invention comprises a method for preparing (meth)acrolein and/or (meth)acrylic acid from an olefin or tert-butanol, the method comprising oxidizing the olefin or tert-butanol in a reactor comprising an inlet for the olefin or tert-butanol to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor has a molar ratio of bismuth to molybdenum that is lower than the catalyst closest to the outlet of the reactor.

### DETAILED DESCRIPTION OF EMBODIMENTS OF THE INVENTION

As used herein, the terms “a,” “an,” “the,” “at least one,” and “one or more” are used interchangeably. The terms “comprises,” “includes,” “contains,” and variations thereof do not have a limiting meaning where these terms appear in the description and claims. Thus, for example, a mixture that includes a polymerization inhibitor can be interpreted to mean that the mixture comprises at least one polymerization inhibitor.

As used herein, the term “(meth)acrolein” refers to both acrolein and methacrolein. Similarly, the term “(meth)acrylic acid” refers to both acrylic acid and methacrylic acid.

As used herein, recitations of numerical ranges by endpoints includes all numbers subsumed in that range (e.g. 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.1 to 100, from 1 to 99.99, from 1.01 to 99.99, from 40 to 6, from 1 to 55, etc.

As used herein, the recitations of numerical ranges and/or numerical values, including such recitations in the claims, can be read to include the term “about.” In such instances, the term “about” refers to numerical ranges and/or numerical values that are substantially the same as those recited herein.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety or its equivalent U.S. version is so incorporated by reference) especially with respect to the disclosure of definitions (to the extent not inconsistent with any definitions specifically provided in this disclosure) and general knowledge in the art.

One aspect of the present invention relates to a reactor for producing (meth)acrolein and/or (meth)acrylic acid from an olefin or tert-butanol. Preferably, the olefin is selected from propylene and isobutylene.

The reactor comprises an inlet for the olefin or tert-butanol to enter the reactor, an outlet for reaction products to exit the reactor, and a catalyst bed disposed between the inlet and the outlet. The catalyst bed comprises a molybdenum and bismuth based mixed oxide catalyst.

As used herein, the term “reaction products” includes the desired products, including (meth)acrolein and/or (meth)acrylic acid, as well as byproducts of the reaction, which may include carbon monoxide, carbon dioxide, acetic acid, formaldehyde, acetaldehyde, propionic acid, propionaldehyde, acetone, allyl alcohol, maleic acid, maleic anhydride, benzaldehyde, benzoic acid, furfural, and phthalic acids, as well as methacrolein/methacrylic acid byproducts including acrolein, acrylic acid, diacetyl, isobutyraldehyde, isobutyric acid, phthalic anhydride, and benzo tricarboxylic acids.

Preferably, the molybdenum and bismuth based mixed oxide catalyst comprises a compound of Formula I:

Mo₁₂BiₐX_{b}Y_{c}Z_{d}Oₑ Formula I
wherein:
X is at least one element selected from the group consisting of iron, cobalt, and nickel;
Y is at least one element selected from the group consisting of magnesium, calcium, strontium, barium, manganese, copper, zinc, cerium, boron, phosphorus, arsenic, tellurium, antimony, chromium, tungsten, sodium, potassium, rubidium, and cesium;
Z is at least one element selected from the group consisting of silicon, aluminum, titanium, tin, and zirconium;
a=0.1 to 2.0;
b=1 to 12;
c=0.01 to 3;
d=0 to 20; and
e is based on the oxidation states of the other elements in the catalyst.

One objective of the present invention is to minimize the formation of byproducts. Byproducts of the reaction are typically removed via distillation, extraction, melt crystallization, or a combination thereof. The byproducts not only consume valuable reactant feed, but also affect the recovery of (meth)acrylic acid and the need to remove the byproducts to prepare certain grades of (meth)acrylic acid. For example, during distillation of crude acrylic acid to make overhead acrylic acid with reduced amounts of maleic anhydride or maleic acid, some acrylic acid must be left in the column bottom along with a majority of the maleic anhydride or maleic acid. For flocculant grade glacial acrylic acid, that limits the amount of furfural or benzaldehyde below 1 ppm, chemical scavengers such as m-phenylenediamine may need to be added to the crude acrylic acid to form higher boiling point adducts with high boiling point aldehydes that are removed in an additional step.

Without wishing to be bound by theory, it is believed that heavy byproducts are related to the formation of the intermediate 1,5-hexadiene. 1,5-Hexadiene is formed as an intermediate by-product when propylene is deprotonated to form a allyl radical that may then react with another allyl radical to form the 1,5-hexadiene. This reaction competes with the main reaction of the allyl radical to form acrolein.

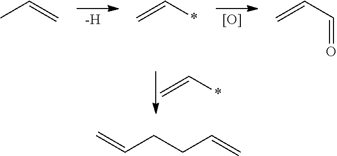

It is further believed that the formation of the allyl radical from propylene is primarily catalyzed by the bismuth in the molybdenum and bismuth based mixed oxide catalyst, and that the formation of acrolein from the allyl radical is primarily catalyzed by the molybdenum. Therefore, to increase the likelihood that the allyl radical will form acrolein, the molar ratio of bismuth to molybdenum is lower in the catalyst closest to the inlet of the reactor than the molar ratio of bismuth to molybdenum in the catalyst closest to the outlet of the reactor. The molar ratio of bismuth to molybdenum may increase from the inlet to the outlet of the reactor to drive propylene conversion to 95% or higher when propylene concentration becomes lower near the outlet of the reactor. In the catalyst, the ratios of other elements may also differ throughout the catalyst bed so that the activity of the catalyst closest to the reactor outlet is equal or higher than the catalyst closest to the inlet of the reactor.

Similarly, it is believed that combinations of the radicals formed in the oxidation reaction of isobutylene or tert-butanol to form methacrolein are also the cause of byproduct formation, and further, having a lower molar ratio of bismuth to molybdenum in the catalyst nearest the inlet will increase the likelihood that the radical formed from isobutylene or tert-butanol will react with the molybdenum to form methacrolein.

The molybdenum and bismuth based mixed oxide catalyst of the catalyst bed may comprise a gradient composition of molybdenum and bismuth in the catalyst. In the gradient composition, the catalyst closest to the inlet of the reactor has a molar ratio of bismuth to molybdenum that is lower than the catalyst closest to the outlet of the reactor. The gradient composition may change linearly between the inlet and the outlet or may change non-linearly between the inlet and the outlet of the reactor. For example, the molar ratio of bismuth to molybdenum may stay relatively constant near the inlet of the reactor and then rapidly increase towards the outlet.

Alternatively, the catalyst bed may comprise two or more zones within the molybdenum and bismuth based mixed oxide catalyst, wherein each zone has a constant molar ratio of bismuth to molybdenum within the zone. The molar ratio of bismuth to molybdenum in the zone closest to the inlet is lower than the molar ratio of bismuth to molybdenum in the zone closest to the outlet of the reactor.

The catalyst bed may comprise three or more zones within the molybdenum and bismuth based mixed oxide catalyst. When the catalyst bed comprises three or more zones within the molybdenum and bismuth based mixed oxide catalyst, the catalyst in the zone or zones between the zone closest to the inlet of the reactor and the zone closest to the outlet of the reactor may have a molar ratio of bismuth to molybdenum that is greater than the molar ratio of bismuth to molybdenum in the catalyst located in the zone closest to the inlet and less than the molar ratio of bismuth to molybdenum in the catalyst in the zone located closest to the outlet of the reactor.

Preferably, the catalyst bed comprises two or more zones within the molybdenum and bismuth based mixed oxide catalyst.

Preferably, the molar ratio of bismuth to molybdenum in the molybdenum and bismuth based mixed oxide catalyst ranges from 0.2:12 to 1.6:12.

Preferably, the molar ratio of bismuth to molybdenum in the catalyst located closest to the inlet of the reactor ranges from 0.2:12 to 1.0:12, and the molar ratio of bismuth to molybdenum in the catalyst located closest to the outlet of the reactor ranges from 0.6:12 to 1.6:12. More preferably, the molar ratio of bismuth to molybdenum in the catalyst located closest to the inlet of the reactor ranges from 0.3:12 to 0.8:12, and the molar ratio of bismuth to molybdenum in the catalyst located closest to the outlet of the reactor ranges from 0.8:12 to 1.4:12.

Another aspect of the present invention relates to a method for preparing (meth)acrolein from a reactant selected from an olefin (e.g., propylene or isobutylene) or tert-butanol comprising oxidizing the reactant in a reactor comprising an inlet for introducing the reactant to the reactor, an outlet for reaction products to exit the reactor, and a catalyst bed disposed between the inlet and the outlet of the reactor. The catalyst bed comprises a molybdenum and bismuth based mixed oxide catalyst, as described above, wherein the catalyst located closest to the inlet of the reactor has a lower bismuth to molybdenum molar ratio than the catalyst located closest to the outlet of the reactor. The method is performed in the gas phase in the presence of oxygen.

In a second step, the (meth)acrolein in the reaction products may be selectively oxidized over a second mixed metal oxide catalyst in the presence of oxygen in the vapor phase, wherein the second mixed metal oxide catalyst has a different composition from the molybdenum and bismuth based mixed oxide catalyst.

The second mixed metal oxide catalyst may be, for example, a solid catalyst that comprises oxides of molybdenum (Mo) and vanadium (V) and/or phosphorus (P). The second mixed metal oxide catalyst may also contain at least one additional element selected from tungsten (W), copper (Cu), iron (Fe), antimony (Sb). When the second mixed metal oxide catalyst contains at least one additional element, the molybdenum and vanadium or phosphorous are the main elements present. Preferably, the second mixed metal oxide catalyst comprises at least 40 wt. % of molybdenum and vanadium and/or phosphorous based on the total weight of non-oxygen elements in the second mixed metal oxide catalyst, such as, for example, at least 50 wt. %, at least 60 wt. %, or at least 70 wt. %.

The second mixed metal oxide catalyst can be any commercially available mixed metal oxide catalyst used for oxidation of (meth)acrolein to (meth)acrylic acid.

In the selective oxidation reaction to form (meth)acrolein and/or (meth)acrylic acid, the oxygen can be present in the form of purified oxygen, oxygen in air, or lattice oxygen of the mixed metal oxide catalyst. Preferably, the oxygen is from air or the lattice oxygen of the mixed metal oxide catalyst.

Purification of the (meth)acrolein and/or (meth)acrylic acid can be achieved by one or more techniques known in the art, such as, for example, absorption using water or an organic solvent, extraction, fractional distillation, or melt crystallization.

### Prophetic Example

The following prophetic example illustrates the present invention but is not intended to limit the scope of the invention.

In a tubular reactor, a catalyst bed will be placed between an inlet of the reactor and an outlet of the reactor. The catalyst bed will be composed of a molybdenum and bismuth based mixed oxide catalyst comprising two zones. The composition of the molybdenum and bismuth based mixed oxide catalyst will be the same in both zones with the exception that the amount of bismuth will be changed. In the first zone closest to the inlet of the reactor, the molar ratio of bismuth to molybdenum will be 0.6:12; and in the second zone closest to the outlet of the reactor, the molar ratio of bismuth to molybdenum will be 1.2:12.

A reactant gas stream comprising 8.1 vol % propylene and 14.4 vol % oxygen will be introduced to the inlet of the reactor.

It is expected that the amount of maleic anhydride will be significantly decreased, e.g., by as much as 50%, compared to commercially available catalysts for the oxidation of propylene to form acrolein.

We claim:

1. A reactor for producing (meth)acrolein and/or (meth)acrylic acid from a reactant selected from propylene, isobutylene and tert-butanol, the reactor comprising an inlet for the reactant to enter the reactor, an outlet for reaction products to exit the reactor, and a catalyst bed disposed between the inlet and the outlet, wherein the catalyst bed comprises a molybdenum and bismuth based mixed oxide catalyst, wherein the catalyst closest to the inlet of the reactor has a molar ratio of bismuth to molybdenum that is lower than the catalyst closest to the outlet of the reactor.

2. The reactor according to claim 1, wherein the catalyst bed comprises a gradient composition of molybdenum and bismuth in the catalyst, wherein the molar ratio of bismuth to molybdenum increases from the catalyst closest to the inlet of the reactor to the catalyst closest to the outlet of the reactor.

3. The reactor according to claim 1, wherein the catalyst bed comprises two or more zones within the catalyst and the catalyst has a different composition within each zone, wherein the catalyst located in the zone closest to the inlet of the reactor has a molar ratio of bismuth to molybdenum that is lower than the catalyst located in the zone closest to the outlet of the reactor.

4. The reactor according to claim 3, wherein the catalyst bed comprises three or more zones within the catalyst, wherein the catalyst located in the zone or zones between the zone closest to the inlet of the reactor and the zone closest to the outlet of the reactor have a molar ratio of bismuth to molybdenum that is greater than the molar ratio of bismuth to molybdenum in the catalyst located in the zone closest to the inlet and less than the molar ratio of bismuth to molybdenum in the catalyst located in the zone closest to the outlet.

5. The reactor according to claim 1, wherein the molybdenum and bismuth based mixed oxide catalyst comprises a compound of Formula I:
Mo₁₂BiₐX_{b}Y_{c}Z_{d}Oₑ Formula I
wherein:
X is at least one element selected from the group consisting of iron, cobalt, and nickel;
Y is at least one element selected from the group consisting of magnesium, calcium, strontium, barium, manganese, copper, zinc, cerium, boron, phosphorus, arsenic, tellurium, antimony, chromium, tungsten, sodium, potassium, rubidium, and cesium;
Z is at least one element selected from the group consisting of silicon, aluminum, titanium, tin, and zirconium;
a=0.1 to 2.0;
b=1 to 12;
c=0.01 to 3;
d=0 to 20; and
e is based on the oxidation states of the other elements in the catalyst.

6. The reactor according to claim 1, wherein the molar ratio of bismuth to molybdenum in the catalyst bed ranges from 0.2:12 to 1.6:12.

7. The reactor according to claim 1, wherein the molar ratio of bismuth to molybdenum in the catalyst located closest to the inlet of the reactor ranges from 0.2:12 to 1.0:12, and the molar ratio of bismuth to molybdenum in the catalyst located closest to the outlet of the reactor ranges from 0.6:12 to 1.6:12.

8. The reactor of claim 7, wherein the molar ratio of bismuth to molybdenum in the catalyst located closest to the inlet of the reactor ranges from 0.3:12 to 0.8:12, and the molar ratio of bismuth to molybdenum in the catalyst located closest to the outlet of the reactor ranges from 0.8:12 to 1.4:12.

9. A method comprising:
oxidizing a reactant selected from propylene, isobutylene and tert-butanol in a reactor according to claim 1, wherein the reaction products exiting the reactor comprise (meth)acrolein.

10. The method according to claim 9, wherein the reactant is propylene and the reaction product comprises acrolein.

11. The method according to claim 10, further comprising oxidizing the acrolein to produce acrylic acid.

12. The method according to claim 9, wherein the reactant is selected from isobutylene and tert-butanol and the reaction product comprises methacrolein.

13. The method according to claim 12, further comprising the methacrolein to product acrylic acid.
